(19) United States
(12) Patent Application Publication
(10) Pub. No.: US 2025/0359885 A1
(43) Pub. Date: Nov. 27, 2025

(54) SURGICAL DEVICE AND METHOD OF USE

(71) Applicant: SUNSURG LLC, Rancho Santa Fe, CA (US)

(72) Inventor: Devin S. ZARKOWSKY, Rancho Santa Fe, CA (US)

(21) Appl. No.: 18/673,045

(22) Filed: May 23, 2024

(51) Int. Cl. A61B 17/32; A61B 17/00; A61B 17/064; A61B 17/068

(52) U.S. Cl. CPC A61B 17/32; A61B 17/064; A61B 17/068; A61B 2017/00141; A61B 2017/00199; A61B 2017/00367; A61B 2017/00818; A61B 2017/00831; A61B 2017/320052; A61B 2017/320064

(57) ABSTRACT

A surgical method includes providing a resection device having a first part and a second part that is spaced apart from the first part to define an opening therebetween. A material is positioned within the opening. The second part is translated relative to the first part to compress the material. Energy is continuously applied to the first part and the second part. The second part is rotated relative to the first part while applying energy to the first part and the second part to cauterize the material, thereby translating linearly along the material's surface cauterizing the material in a continuous fashion. In some embodiments, devices and systems are disclosed.

### TECHNICAL FIELD

The present disclosure generally relates to medical devices for the treatment via cautery and stapling—separately or in combination—followed by resection of human tissues, including but not limited to visceral, muscular, mesenteric, or integumentary. Surgical scenarios requiring application of this device include but are not limited to trauma, intestinal obstruction, cancer, and other organ dysfunction as deemed appropriate by the treating physician.

### BACKGROUND

An example use case for a surgical resection device includes small bowel resection in elective and emergent surgical situations with myriad indications, including trauma, cancer, obstruction, and infarction. Each procedure necessitates different technical elements, but the tools available for tissue resection all mimic clamp action. Surgeons target tissue, create a landing zone for the clamp or clamp-like energy tool and engage the device; this process is somewhat cumbersome and can require 10 to 45 minutes, or about 15 to 45% of the total operative time, depending on the procedure and length of bowel intended for resection.

The Healthcare Cost and Utilization Project estimates 1,302,900 patients received operations involving total or partial organ resection in 2018 (McDermott K W, Liang L. Overview of Operating Room Procedures During Inpatient Stays in US Hospitals, 2018. 2021; https://hcup-us.ahrq.gov/reports/statbriefs/sb281-Operating-Room-Procedures-During-Hospitalization-2018.pdf. Accessed Mar. 1, 2024). The Dutch COLOR Study Group correlated case volume, operating time and outcome in a 2005 paper focused on large bowel resection (Kuhry E, Bonjer H J, Haglind E, et al. Impact of hospital case volume on short-term outcome after laparoscopic operation for colonic cancer. Surg Endosc. 2005;19(5):687-692). Patients operated on by high-volume surgeons able to perform procedures in less time demonstrated statistically superior intraoperative and postoperative outcomes, as well as shorter hospital stays. Findings suggesting that shorter operative times lead to improved outcomes have been recapitulated in published literature for multiple other surgical sub-specialties.

Both open and laparoscopic resection techniques revolve around three basic steps: specimen isolation, blood supply ligation, and alimentary tract refunctionalization. Surgeons select their preferred tools—including clamps, suture, staplers, and energy devices—to complete these steps based on training and surgical exposure. Another publication incorporating cost feedback mechanisms to influence surgeon preference, demonstrated improved outcomes and decreased expenditures with attention to this element of surgical care (Zygourakis C C, Valencia V, Moriates C, et al. Association Between Surgeon Scorecard Use and Operating Room Costs. JAMA Surg. 2016). In terms of equipment, the most cost-effective combination includes bowel clamps, crushing surgical clamps, and suture. Capital and recurrent costs associated with the reusable clamps vary by institution, but have been estimated at around $0.51 per instrument, usually as part of approximately 100 instruments in a tray (Stockert E W, Langerman A. Assessing the magnitude and costs of intraoperative inefficiencies attributable to surgical instrument trays. J Am Coll Surg. 2014;219(4):646-655). Suture material employed in blood vessel ligation and either bowel anastomosis or ostomy creation lists for $6.00 per pack, with 3 to 8 packs required to complete the procedure. Alternatively, a single stapling device may both transect and reconnect bowel, and in some cases, divide the associated blood supply. The stapling handle plus one staple load costs $135at the applicant's clinical institution, with reloads costing $72 each; a minimum of 4 staple fires is required for a stapled resection and anastomosis. Time necessary to resect bowel with these tools varies by the length of the specimen; anywhere from 15 minutes to 60 minutes may be required. Valuing this time in the United States is a complex process governed by byzantine accounting methods associated with cost-to-charge ratios that vary between institutions (Macario A. What does one minute of operating room time cost? J Clin Anesth. 2010;22(4):233-236). Specific dollar values per minute of OR time depend on procedure length and complexity, ranging between $22 and $133 per minute (Macario A. What does one minute of operating room time cost? J Clin Anesth. 2010;22(4):233-236). Reduced operating time improves patient outcomes while decreasing expenditures by the health care system (Kuhry E, Bonjer H J, Haglind E, et al. Impact of hospital case volume on short-term outcome after laparoscopic operation for colonic cancer. Surg Endosc. 2005;19(5):687-692 and Aziz F, Lehman E B, Reed A B. Increased Duration of Operating Time for Carotid Endarterectomy Is Associated with Increased Mortality. Ann Vasc Surg. 2016;36:166-174); for this reason, energy devices evolved in the 2000's to decrease procedure length. These clamp-like instruments cauterize tissue between bipolar electrical leads or with heat generated from ultrasonic vibration (Chen X L, Chen X Z, Lu Z H, et al. Comparison of ultrasonic scalpel versus conventional techniques in open gastrectomy for gastric carcinoma patients: a systematic review and meta-analysis. PLOS One. 2014;9(7):e103330 and Di Lorenzo N, Franceschilli L, Allaix M E, Asimakopoulos A D, Sileri P, Gaspari A L. Radiofrequency versus ultrasonic energy in laparoscopic colorectal surgery: a metaanalysis of operative time and blood loss. Surg Endosc. 2012;26(10):2917-2924). It has been found that the time required to transition from tissue target acquisition to complete energy delivery to the next target acquisition is 18 seconds; the rate of tissue cautery depends on both the jaw length of the instrument-which varies between 1 and 3 cm for current commercially-available devices-and the length of tissue resected. Besides small and large bowel resection, these devices are employed in thyroid, esophagus, lung, stomach, pancreas, liver, kidney, spleen and rectal procedures. This disclosure describes an improvement over prior technologies.

### SUMMARY

In one embodiment, in accordance with the principles of the present disclosure, a resection device comprises a first part and a second part that is rotatable relative to the first part, the second part being spaced apart from the first part to define an opening therebetween. The second part is configured to translate relative to the first part to compress a material positioned within the opening. The first part and the second part are configured to continuously receive electrical energy to cauterize the material as the second part rotates relative to the first part.

In one embodiment, in accordance with the principles of the present disclosure, a surgical method comprises providing a resection device, wherein the resection device comprises a first part and a second part that is spaced apart from the first part to define an opening therebetween. A material is positioned within the opening. The second part is translated relative to the first part to compress the material. Electrical energy is continuously applied to the first part and the second part. The second part is rotated relative to the first part while applying electrical energy to the first part and the second part to cauterize the material.

In one embodiment, in accordance with the principles of the present disclosure, a surgical method comprises providing a resection device. The resection device comprises a first part and a second part. The first part includes a first pair of rollers and a first tread extending about the first pair of rollers. The second part includes a second pair of rollers and a second tread extending about the second pair of rollers. The second part is spaced apart from the first part to define an opening therebetween. A material is positioned within the opening. The second part is translated relative to the first part to compress the material. Electrical energy is continuously applied to the first part and the second part. The second part is rotated relative to the first part while applying electrical energy to the first part and the second part to cauterize the material. The material includes, for example, a first section of a bowel and a second section of the bowel. Cauterizing the material seals the first section of the bowel with the second section of the bowel. The second part is translated relative to the first part to compress the material with a force of about 100 N to about 300 N. The electrical energy that is continuously applied is produced by an electrical conditioning and supply unit.

In one embodiment, in accordance with the principles of the present disclosure, without relinquishing the clamped state wherein the material is compressed within the opening, but adjusting the amount of pressure or compression applied by further translating the second part relative to the first part as needed, the first and second parts translate linearly across an organ surface, applying electrical energy in a continuous fashion to the material (tissue) captured between the first part and the second part. That is, the tissue may be provisionally compressed between the first and second parts by translating the second part relative to the first part in a first direction. After the tissue is provisionally compressed, the second part may be translated relative to the first part in an opposite second direction to decrease the amount of force applied to the tissue and allow the first and second parts translate linearly across the organ surface, while continuously applying electrical energy. As used in this example, tissue may include, for example, further sections of bowel or the mesentery tissue containing blood vessels and lymphatic tissue associated with the bowel. It is envisioned that the linear translation of the first part and second part across the organ surface may be accomplished by the treating physicians' hands, a power drive train causing the first part and second part to rotate relative to each other, or a combination of both.

### BRIEF DESCRIPTION OF THE DRAWINGS

The present disclosure will become more readily apparent from the specific description accompanied by the following drawings, in which: Target tissue is treated with energy derived from multiple sources.

FIG. 1 is a perspective view, in part phantom, of one embodiment of a continuous resection device of a surgical system, in accordance with principles of the present disclosure;

FIG. 2 is a perspective view, in part phantom, of a portion of the resection device shown in FIG. 1, with parts of the resection device in a first position;

FIG. 3 is a perspective view, in part phantom, of the portion of the resection device shown in FIG. 2, with the parts of the resection device in a second position;

FIG. 4 is a side view, in part phantom, of a portion of the resection device shown in FIG. 1, with the parts of the resection device in the first position;

FIG. 5 is a side view, in part phantom, of the portion of the resection device shown in FIG. 4, with the parts of the resection device in the second position;

FIG. 6 is a plan view of the resection device shown in FIG. 1, together with other components of the surgical system, in accordance with principles of the present disclosure;

FIG. 7 is a plan view of the resection device shown in FIG. 1 in use with a sample material, in accordance with principles of the present disclosure;

FIG. 8 is a plan view of the resection device shown in FIG. 1 in use with the sample material shown in FIG. 7, in accordance with principles of the present disclosure;

FIG. 8A is a plan view of the sample material shown in FIG. 7, in accordance with principles of the present disclosure;

FIG. 8B is a plan view of the sample material shown in FIG. 7, in accordance with principles of the present disclosure;

FIG. 8C is a plan view of the sample material shown in FIG. 7, in accordance with principles of the present disclosure;

FIG. 8D is a plan view of the sample material shown in FIG. 7, in accordance with principles of the present disclosure;

FIG. 9 is a perspective view, in part phantom, of a portion of one embodiment of a resection device of the surgical system, in accordance with principles of the present disclosure;

FIG. 10 is a perspective view, in part phantom, of the portion of the embodiment of the resection device shown in FIG. 9, coupled with another component of the surgical system, in accordance with principles of the present disclosure;

FIG. 11 is a perspective view of one embodiment of a resection device of the surgical system, in accordance with principles of the present disclosure;

FIG. 12 is a plan view, in part phantom, of the resection device shown in FIG. 11;

FIG. 13 is a perspective view, in part phantom, of the resection device shown in FIG. 11; and in FIG. 11.

FIG. 14 is a plan view showing a function of the resection device shown

Like reference numerals indicate similar parts throughout the figures.

### DETAILED DESCRIPTION

The exemplary embodiments of a surgical system and related methods of use disclosed are discussed in terms of medical devices for the treatment of tissue resection in a continuous manner, and more particularly but not exclusively, in terms of small bowel, large bowel, thyroid, esophagus, lung, stomach, pancreas, liver, kidney, spleen, and rectal procedures. In some embodiments, other medical procedures involving various anatomy is contemplated.

In one embodiment, in accordance with the principles of the present disclosure, the surgical system includes an in-line, continuous resection instrument that is configured to decrease operative time and cost in comparison to conventional devices, such as, for example, clamps and current clamp-derived devices. Furthermore, it is believed that the disclosed continuous resection instrument can function to seal bowel lumen and/or bowel mesentery in a manner that is at least functionally equivalent to standard techniques using conventional devices, and in some cases, improves upon results obtained using standard techniques and conventional devices.

The disclosed continuous resection instrument is configured to cauterize tissue in an in-line fashion. Resection may or may not then be accomplished by interaction with a fixed or movable blade. One iteration, the disclosed continuous resection instrument connects to electrical power, which is subsequently transferred to tissue by a pair of two-wheel drive-trains. These drive trains may or may not compress target tissue between electrically conductive “treads” as electrical power is applied. The spacing between the treads can be adjusted to accommodate thicker tissue and to pass over the target tissue in order to interact with the tissue to be ligated and cut. That is, the treads can be translated relative to one another in opposite directions to facilitate target tissues of different thicknesses and to selectively increase and/or decrease the amount of force or compression that is applied to the target tissue by the treads. For example, in some embodiments, the treads may be translated relative to one another to compress the target tissue with a first amount of force; the treads may be subsequently translated relative to one another in an opposite direction such that the treads compress the target tissue with a decreased second amount of force to allow the treads to translate linearly across an organ surface, as discussed herein.

The rate of passage from the treads to the cutting element (fixed or movable blade) will be determined by monitoring the change in power applied to the treated length of tissue, as well as the tissue temperature. That is, the system of the present disclosure can be adapted to determine if and when the tissue has been sufficiently cauterized. For example, in some embodiments, the disclosed continuous resection instrument can be adapted to function with monitoring equipment that is adapted to provide continuously-updated power and treated tissue temperature information. This information may be displayed via a screen readout mounted on an electrical conditioning and supply unit to which the continuous resection device will be connected, as described in greater detail hereinbelow.

In one embodiment, the disclosed continuous resection instrument can be adapted to function with laser energy. The drive train rollers may or may not compress the tissue, as discussed above. However, when laser energy is used with the disclosed device, the laser energy can be focused on the target tissue between the drive train rollers as the tissue is compressed between the rollers and/or after the rollers translate across the tissue to resection or dissociate the tissue. It is envisioned that the laser energy may be focused on one or both sides of the target tissue. In one example, tissue dissociation may be accomplished using the fixed or movable blade and/or laser energy, depending on the requirements of a particular application.

In on embodiment, the disclosed continuous resection instrument can be adapted to function with staples. The drive train rollers can be moved over the target tissue in continuous fashion, as discussed above. Staples may be placed in one or more rows on either or both sides of the target tissue, prior to, during, or after the drive train rollers are moved over the target tissue; electrical or laser energy may or may not be applied in addition to the staples. The target tissue may then be ligated or cut by a fixed or movable blade.

In some embodiments, the disclosed continuous resection instrument cauterizes in an in-line, continuous fashion, eliminating ancillary medical devices and decreasing the time required for surgical resections. By contrast, current commercially-available devices all mimic clamp action and do not have an Instructions For Use (IFU) indication that includes bowel cauterization. Conventional devices combine cauterization with a cutting function, require a surgeon to target tissue, grasp and lock the device, engage the cautery function and then release over 10's of centimeters. This repetitive process consumes significant operating room time. Targeting specimen tissue, reliably cauterizing and sealing, as well as transecting the blood supply in an in-line, continuous fashion using the disclosed continuous resection instrument will expedite procedures by eliminating the step-wise process forced by current clamp-like energy instruments. Rather than altering the course of procedures, the continuous resection instrument of the present disclosure will streamline established practices with negligible adjustment by the surgeon.

The disclosed continuous resection instrument incorporates two important concepts. First, common operating room cautery parameters meet large resistances (>10 kOhms) when applied to intestinal tissue lengths greater than 120 mm (˜25 mm² cross section). This limits the distance between the active electrodes that can be used in any radiofrequency electrosurgical device since the impedance presented to the electrodes will limit the power that can be transferred to the tissue. Accordingly, monopolar systems are not viable options to effectively seal subsurface vessels evenly since the return electrode is usually quite distant. In order to design an effective sealing device, the disclosed continuous resection instrument employs an electrical conditioning and supply unit that will provide energy to seal, for example, bowel lumen which is approximately 20 mm thick. Clamping will further reduce this thickness to a few millimeters. Second, the disclosed continuous resection instrument is configured to apply a clamping force of ˜100 to 300 N between drive train rollers of the device to reliably seal bowel lumen and vessels, as discussed herein. The drive train rollers can be adapted to establish and maintain a contact force between 100 and 300 N between the rollers. The disclosed continuous resection instrument can implement a compression mechanism that will maintain this force between the drive train rollers as the device translates linearly across tissue and applies energy to cauterize and/or seal the target tissue.

In some embodiments, one or all of the components of the surgical system may include disposable, peel-pack, pre-packed sterile devices. In some embodiments, the components of the surgical system are configured for one-time use and are disposed after they are used one time. However, it is contemplated that one or all of the components of the surgical system may be reusable. In some embodiments, one or more of the components of the surgical system are configured to be sterilized.

In some embodiments, the disclosed packages, surgical methods, and systems may be alternatively employed in a surgical treatment with a patient in a prone or supine position, and/or employ various surgical approaches, including open incisions, laparoscopic, or robotic in any body region. The packages, implants, methods and systems of the present disclosure may also be used on animals, tissue models, and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions, or parameters described and/or shown herein, and that the terminology used herein is for the purpose of describing particular embodiments by way of example only and is not intended to be limiting of the claimed disclosure. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior.”

Further, as used in the specification and including the appended claims, “treating” or “treatment” of a disease or condition refers to performing a procedure to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic obstruction; as an adjunct in surgery; and/or any repair procedure. Also, as used in the specification and including the appended claims, the term “tissue” includes soft tissue, ligaments, tendons, cartilage, nerves, brain matter, blood vessels, muscle, and/or bone unless specifically referred to otherwise.

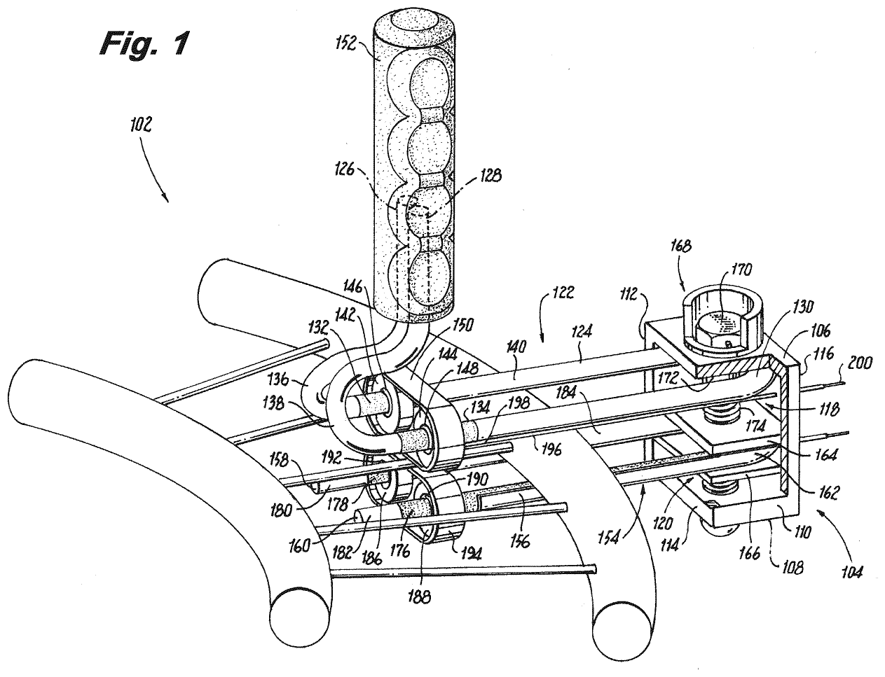

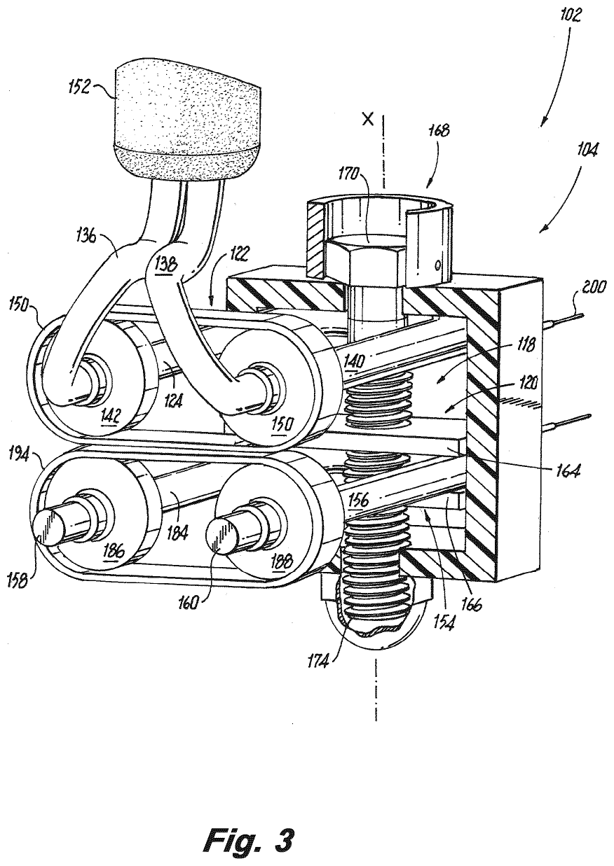

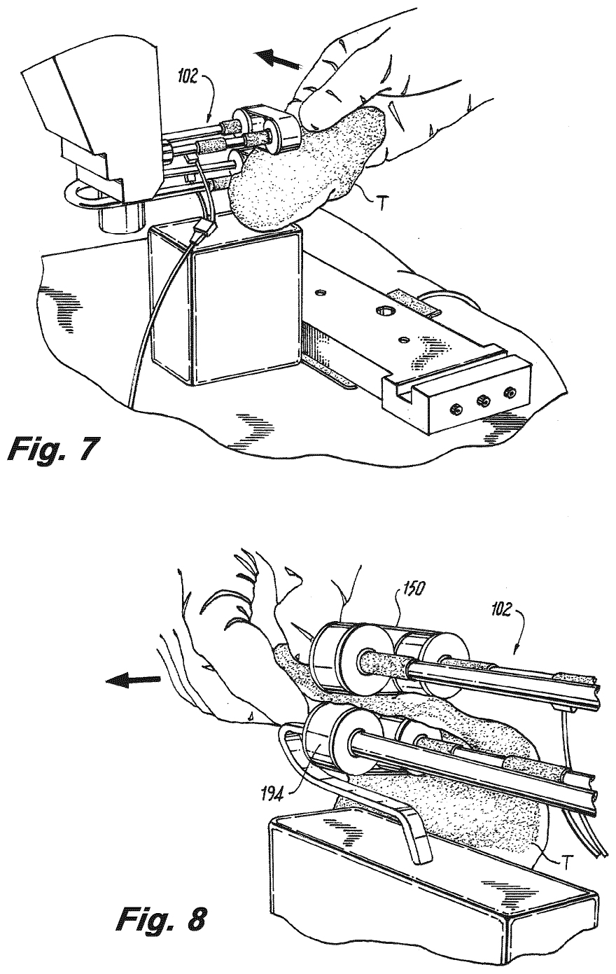

The following discussion includes a description of a surgical system and related methods of employing the system in accordance with the principles of the present disclosure. Alternate embodiments are also disclosed. Reference will now be made in detail to the exemplary embodiments of the present disclosure, which are illustrated in the accompanying figures. Turning now to FIGS. 1-14, there are illustrated components of a surgical system 100 in accordance with the principles of the present disclosure.

The components of surgical system 100 can be fabricated from biologically acceptable materials suitable for medical applications, including metals, synthetic polymers, ceramics, and/or their composites, depending on the particular application and/or preference of a medical practitioner. For example, the components of surgical system 100, individually or collectively, can be fabricated from materials such as thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of surgical system 100 may have material composites, including the above materials, to achieve various desired characteristics such as strength, rigidity, elasticity, compliance, biomechanical performance, durability and radiolucency or imaging preference. The components of surgical system 100, individually or collectively, may also be fabricated from a heterogeneous material such as a combination of two or more of the above-described materials. The components of surgical system 100 may be monolithically formed, integrally connected or include fastening elements and/or instruments, as described herein.

In one embodiment, in accordance with the principles of the present disclosure, surgical system 100 includes an in-line, continuous resection instrument, such as, for example, a resection device 102. Resection device 102 is configured to cauterize tissue in an in-line fashion by compressing the tissue, translating linearly across the tissue and applying electrical energy in a continuous fashion to cauterize and/or seal the tissue, as discussed herein. Resection device 102 includes a housing 104 configured for engagement with components of resection device 102 in a manner that allows relative translation between the components, as discussed herein. Housing 104 includes a top wall 106 and an opposite bottom wall 108, as shown in FIGS. 1-3, for example. Housing 104 further includes a first side wall 110, a second side wall 112 opposite first side wall 110, a first end wall 114 and a second end wall 116 opposite first end wall 114. First side wall 110, second side wall 112, first end wall 114 and second end wall 116 each extend continuously from top wall 106 to bottom wall 108. Inner surfaces of top wall 106, bottom wall 108, first side wall 110, second side wall 112, first end wall 114 and second end wall 116 define a cavity 118 of housing 104. Housing 104 includes an opening 120 extending through first end wall 114. Opening 120 is in communication with cavity 118. In some embodiments, housing 104 is monolithically and/or integrally formed. In some embodiments, cavity 118 and/or opening 120 may have various cross section configurations, such as, for example, oval, oblong, triangular, rectangular, square, polygonal, irregular, uniform, non-uniform, variable, tubular and/or tapered.

Resection device 102 includes a first part, such as, for example, a first roller assembly 122. First roller assembly 122 includes an extension, such as, for example, a shaft 124. Shaft 124 includes a first end 126, a second end 128 opposite first end 126, and an intermediate portion 130 positioned between first end 126 and second end 128. Intermediate portion 130 extends through opening 120 for disposal within cavity 118. In some embodiments, intermediate portion 130 includes an arcuate portion that defines a loop that is positioned within cavity 118. In some embodiments, intermediate portion 130 is positioned within cavity 118 such that intermediate portion 130 is fixed relative to housing 104 to prevent relative movement between shaft 124 and housing 104. In some embodiments, intermediate portion 130 of shaft 124 can be variously connected with housing 104, such as, for example, monolithic, integral connection, frictional engagement, threaded engagement, mutual grooves, screws, adhesive, nails, barbs, raised elements, spikes, clips, snaps, friction fittings, compressive fittings, expanding rivets, staples, fixation plates, key/keyslot, tongue in groove, dovetail, magnetic connection and/or posts. In some embodiments, shaft 124 is monolithically and/or integrally formed.

Shaft 124 includes a first conductive portion 132 between first end 126 and intermediate portion 130, and a second conductive portion 134 between second end 128 and intermediate portion 130. Shaft 124 includes a first non-conductive portion 136 between first conductive portion 132 and first end 126, a second non-conductive portion 138 between second conductive portion 134 and second end 128, and a third non-conductive portion 140 between first conductive portion 132 and second conductive portion 134. In some embodiments, first conductive portion 132 and/or second conductive portion 134 is/are made at least in part from a material that is electrically conductive. In some embodiments, first conductive portion 132 and/or second conductive portion 134 is/are made at least in part from a material that is not electrically conductive and a material that is electrically conductive. For example, in some embodiments, first conductive portion 132 and/or second conductive portion 134 can be made from a material that is not electrically conductive and is coated and/or layered with a material that is electrically conductive. In some embodiments, first non-conductive portion 136, second non-conductive portion 138 and/or third non-conductive portion 140 is/are made at least in part from a material that is not electrically conductive. In some embodiments, first non-conductive portion 136, second non-conductive portion 138 and/or third non-conductive portion 140 is/are made at least in part from a material that is electrically conductive and a material that is not electrically conductive. For example, in some embodiments, first non-conductive portion 136, second non-conductive portion 138 and/or third non-conductive portion 140 can be made from a material that is electrically conductive and is coated and/or layered with a material that is not electrically conductive.

First roller assembly 122 includes a wheel, such as, for example, a first roller 142 disposed about first conductive portion 132 of shaft 124 such that shaft 124 extends through first roller 142 and first roller 142 is rotatable relative to shaft 124. That is, first roller 142 is configured to rotate 360 degrees about shaft 124 in opposite directions. First roller assembly 122 further includes a wheel, such as, for example, a second roller 144 disposed about second conductive portion 134 of shaft 124 such that shaft 124 extends through second roller 144 and second roller 144 is rotatable relative to shaft 124. That is, second roller 144 is configured to rotate 360 degrees about shaft 124 in opposite directions. First conductive portion 132 is positioned relative to second conductive portion 134 such that first roller 142 is spaced apart from second roller 144. In some embodiments, first roller 142 and/or second roller 144 are configured to rotate freely relative to shaft 124. That is, first roller 142 and/or second roller 144 are configured to be rotated relative to shaft 124 manually. In some embodiments, first roller 142 and/or second roller 144 are configured to rotate relative to shaft 124 via automation. For example, in some embodiments, roller assembly 122 includes a hub 146 that is disposed about first conductive portion 132 of shaft 124 and first roller 142 is disposed about hub 146. Hub 146 can include an actuator, such as, for example, a motor that is configured to rotate first roller 142 about hub 146 for rotation of first roller 142 about shaft 124. Likewise, in some embodiments, roller assembly 122 includes a hub 148 that is disposed about second conductive portion 134 of shaft 124 and second roller 144 is disposed about hub 148 for rotation of second roller 144 about shaft 124. Hub 148 can include an actuator, such as, for example, a motor that is configured to rotate second roller 144 about hub 148 for rotation of second roller 144 about shaft 124. In some embodiments, first roller 142, second roller 144, hub 146 and/or hub 148 is/are made at least in part from a material that is electrically conductive such that electrical current from first conductive portion 132 and/or the second conductive portion 134 will conduct through first roller 142, second roller 144, hub 146 and/or hub 148, as discussed herein. In some embodiments, first roller 142, second roller 144, hub 146 and/or hub 148 is/are made at least in part from the same material that first conductive portion 132 and/or second conductive portion 134 is/are made from. In some embodiments, first roller 142, second roller 144, hub 146 and/or hub 148 is/are made at least in part from a material that is different than the material that first conductive portion 132 and/or second conductive portion 134 is/are made from.

First roller assembly 122 includes a band, such as, for example, a tread 150 extending about first roller 142 and second roller 144. Tread 150 is configured for rotation relative to shaft 124. That is, tread 150 is positioned about first roller 142 and second roller 144 such that rotation of first roller 142 and second roller 144 relative to shaft 124 also rotates tread 150 relative to shaft 124. Tread 150 is an electrode and is made at least in part from a material that is electrically conductive such that electrical current from the first roller 142 and/or the second roller 144 will conduct through tread 150, as discussed herein. In some embodiments, tread 150 includes electrically conductive tape. In some embodiments, tread 150 includes one or a plurality of layers. In some embodiments, tread 150 has a band or loop configuration and extends continuously about first roller 142 and second roller 144. In some embodiments, tread 150 extends about first roller 142 and second roller 144 in a manner that prevents relative movement between tread 150 and first roller 142 and/or second roller 144. In some embodiments, tread 150, first roller 142 and/or second roller 144 may have various surface configurations to enhance fixation of tread 150 with first roller 142 and/or second roller 144, such as, for example, rough, arcuate, undulating, porous, semi-porous, dimpled, polished and/or textured according to the requirements of a particular application. In some embodiments, tread 150 can be variously connected with first roller 142 and/or second roller 144, such as, for example, monolithic, integral connection, frictional engagement, mutual grooves, adhesive, raised elements, spikes, clips, snaps, friction fittings, compressive fittings, expanding rivets, staples, fixation plates, key/keyslot, tongue in groove, dovetail, magnetic connection and/or posts.

In some embodiments, first end 126 and second end 128 of shaft 124 converge and are joined by a handle 152. Handle 152 is configured for gripping by hand by a medical practitioner while resection device 102 is translating linearly across tissue, for example. As shown in FIGS. 1-3, handle 152 is positioned between first roller 142 and second roller 142 enabling the surgeon to visualize progress from treated tissue to target tissue without visual obstruction, stabilize the embodiment during treatment, and present the treatment mechanism to an assistant facilitating the procedure. In some embodiments, handle 152 can include extruded plastic, for example. In some embodiments, handle 152 is ergonomic. In some embodiments, handle 152 can include one or a plurality of gripping features, such as, for example, grooves or recesses. In some embodiments, handle 152 extends perpendicular to first conductive portion 132, second conductive portion 134 and/or third non-conductive portion 140 of shaft 124. However, it is envisioned that handle 152 may be disposed at alternate orientations, relative to first conductive portion 132, second conductive portion 134 and/or third non-conductive portion 140, such as, for example, transverse and/or other angular orientations such as acute or obtuse, and/or may be offset or staggered.

Resection device 102 includes a second part, such as, for example, a second roller assembly 154. Second roller assembly 154 includes an extension, such as, for example, a shaft 156. Shaft 156 includes a first end 158, a second end 160 opposite first end 158, and an intermediate portion 162 positioned between first end 158 and second end 160. Intermediate portion 162 extends through opening 120 for disposal within cavity 118. In some embodiments, intermediate portion 162 includes an arcuate portion that defines a loop that is positioned within cavity 118. In some embodiments, shaft 156 extends parallel to first conductive portion 132, second conductive portion 134 and/or third non-conductive portion 140 along an entire length of shaft 156. However, it is envisioned that all or part of shaft 156 may be disposed at alternate orientations, relative to first conductive portion 132, second conductive portion 134 and/or third non-conductive portion 140, such as, for example, transverse and/or other angular orientations such as acute or obtuse, and/or may be offset or staggered. In some embodiments, shaft 156 is monolithically and/or integrally formed.

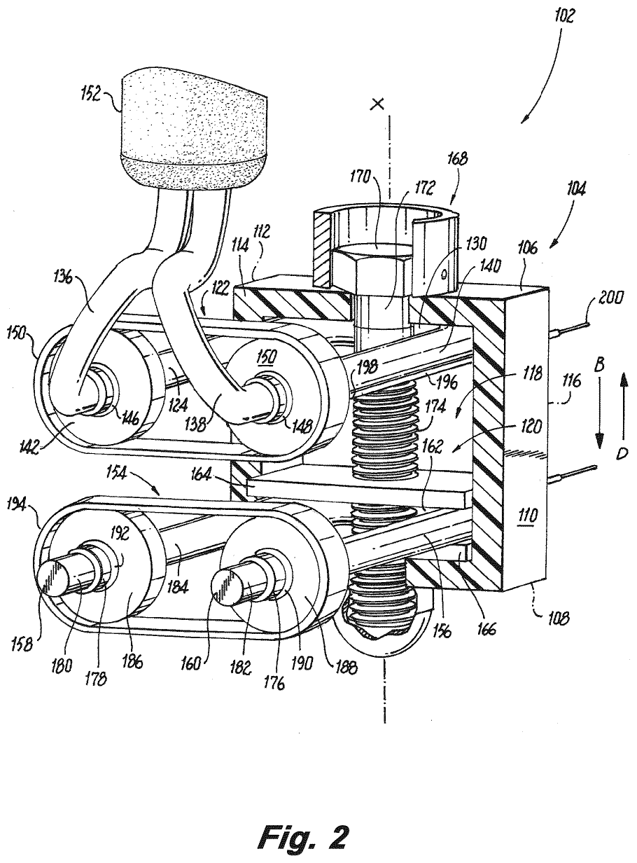

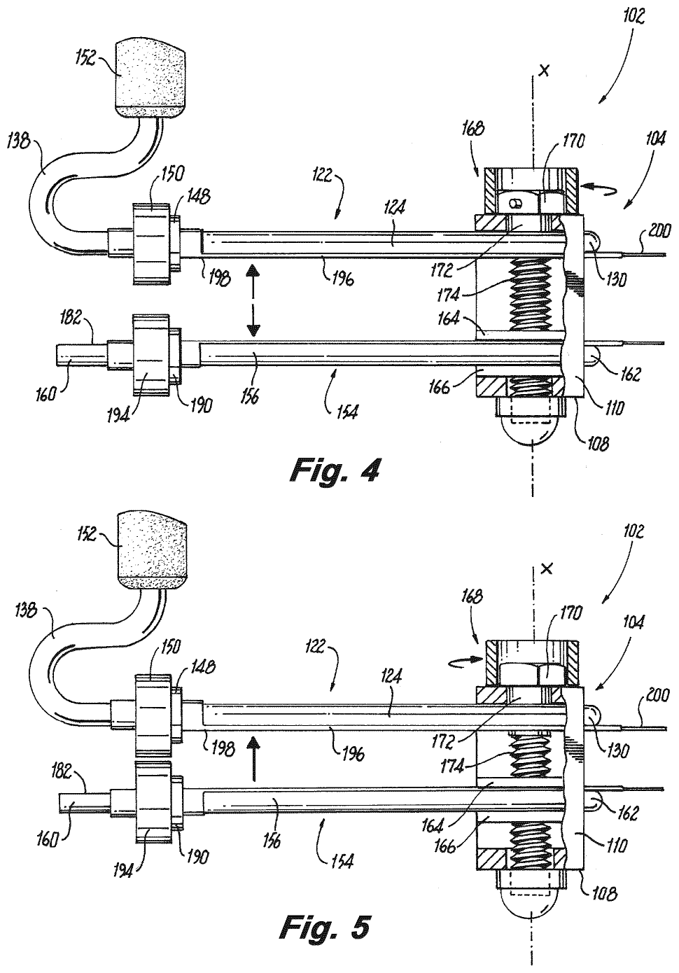

In some embodiments, resection device 102 includes a first part, such as, for example, a first plate 164 and a second part, such as, for example, a second plate 166. First plate 164 and second plate 166 are each movably positioned within cavity 118 of housing 104. In particular, in some embodiments, first plate 164 is positioned within cavity 118 such that first plate 164 directly engages a top or proximal surface of intermediate portion 162 and second plate 166 is positioned within cavity 118 such that second plate 166 directly engages a bottom or distal surface of intermediate portion 162. In some embodiments, the first plate 164 and the second plate 166 maintain the parallel alignment between shaft 156 and first conductive portion 132, second conductive portion 134 and/or third non-conductive portion 140 of shaft 124. Resection device 102 includes an actuator, such as, for example, a screw 168 having a head 170, a non-threaded portion 172 connected to head 170 and a threaded portion 174 connected to non-threaded portion 172. Non-threaded portion 172 extends through top wall 106 of housing and threaded portion 174 extends through first plate 164 and second plate 164 such that rotation of screw 168 relative to housing 104 in a first rotational direction, such as, for example, the direction shown by arrow A in FIG. 4, translates shaft 156 relative to housing 104 and/or shaft 124 in the direction shown by arrow B in FIG. 2 along a longitudinal axis X defined by screw 168; and rotation of screw 168 relative to housing 104 in an opposite second rotational direction, such as, for example, the direction shown by arrow C in FIG. 5, translates shaft 156 relative to housing 104 and/or shaft 124 in the direction shown by arrow D in FIG. 2 along longitudinal axis X.

Shaft 156 includes a first conductive portion 176 between first end 158 of shaft 156 and intermediate portion 162, and a second conductive portion 178 between second end 160 of shaft 156 and intermediate portion 162. Shaft includes a first non-conductive portion 180 between first conductive portion 176 and first end 158 of shaft 156, a second non-conductive portion 182 between second conductive portion 182 and second end 160 of shaft 156, and a third non-conductive portion 184 between first conductive portion 176 and second conductive portion 178. In some embodiments, first conductive portion 176 and/or second conductive portion 178 is/are made at least in part from a material that is electrically conductive. In some embodiments, first conductive portion 176 and/or second conductive portion 178 is/are made at least in part from a material that is not electrically conductive and a material that is electrically conductive. For example, in some embodiments, first conductive portion 176 and/or second conductive portion 178 can be made from a material that is not electrically conductive and is coated and/or layered with a material that is electrically conductive. In some embodiments, first non-conductive portion 180, second non-conductive portion 182 and/or third non-conductive portion 184 is/are made at least in part from a material that is not electrically conductive. In some embodiments, first non-conductive portion 180, second non-conductive portion 182 and/or third non-conductive portion 184 is/are made at least in part from a material that is electrically conductive and a material that is not electrically conductive. For example, in some embodiments, first non-conductive portion 180, second non-conductive portion 182 and/or third non-conductive portion 184 can be made from a material that is electrically conductive and is coated and/or layered with a material that is not electrically conductive.

Second roller assembly 154 includes a wheel, such as, for example, a first roller 186 disposed about first conductive portion 176 of shaft 156 such that shaft 156 extends through first roller 186 and first roller 186 is rotatable relative to shaft 156. That is, first roller 186 is configured to rotate 360 degrees about shaft 156 in opposite directions. Second roller assembly 154 further includes a wheel, such as, for example, a second roller 188 disposed about second conductive portion 178 of shaft 156 such that shaft 156 extends through second roller 188 and second roller 188 is rotatable relative to shaft 156. That is, second roller 188 is configured to rotate 360 degrees about shaft 156 in opposite directions. First conductive portion 176 is positioned relative to second conductive portion 178 such that first roller 186 is spaced apart from second roller 188. In some embodiments, first roller 186 and/or second roller 188 are configured to rotate freely relative to shaft 156. That is, first roller 186 and/or second roller 188 are configured to be rotated relative to shaft 156 manually. In some embodiments, first roller 186 and/or second roller 188 are configured to rotate relative to shaft 156 via automation. For example, in some embodiments, second roller assembly 154 includes a hub 190 that is disposed about first conductive portion 176 of shaft 156 and first roller 186 is disposed about hub 190. Hub 190 can include an actuator, such as, for example, a motor that is configured to rotate first roller 186 about hub 192 for rotation of first roller 186 about shaft 156. Likewise, in some embodiments, second roller assembly 154 includes a hub 192 that is disposed about second conductive portion 178 of shaft 156 and second roller 188 is disposed about hub 192 for rotation of second roller 188 about shaft 156. In some embodiments, first roller 186, second roller 188, hub 190 and/or hub 192 is/are made at least in part from a material that is electrically conductive such that electrical current from first conductive portion 176 and/or the second conductive portion 178 will conduct through first roller 186, second roller 188, hub 190 and/or hub 192, as discussed herein. In some embodiments, first roller 186, second roller 188, hub 190 and/or hub 192 is/are made at least in part from the same material that first conductive portion 176 and/or second conductive portion 178 is/are made from. In some embodiments, first roller 186, second roller 188, hub 190 and/or hub 192 is/are made at least in part from a material that is different than the material that first conductive portion 176 and/or second conductive portion 178 is/are made from.

Second roller assembly 154 includes a band, such as, for example, a tread 194 extending about first roller 186 and second roller 188. Tread 194 is configured for rotation relative to shaft 156. That is, tread 194 is positioned about first roller 186 and second roller 188 such that rotation of first roller 186 and second roller 188 relative to shaft 156 also rotates tread 194 relative to shaft 156. Tread 194 is made at least in part from a material that is electrically conductive such that electrical current from the first roller 186 and/or the second roller 188 will conduct through tread 194, as discussed herein. In some embodiments, tread 194 includes electrically conductive tape. In some embodiments, tread 194 includes one or a plurality of layers. In some embodiments, tread 194 has a band or loop configuration and extends continuously about first roller 186 and second roller 188. In some embodiments, tread 194 extends about first roller 186 and second roller 188 in a manner that prevents relative movement between tread 194 and first roller 186 and/or second roller 188. In some embodiments, tread 194, first roller 186 and/or second roller 188 may have various surface configurations to enhance fixation of tread 194 with first roller 186 and/or second roller 188, such as, for example, rough, arcuate, undulating, porous, semi-porous, dimpled, polished and/or textured according to the requirements of a particular application. In some embodiments, tread 194 can be variously connected with first roller 186 and/or second roller 188, such as, for example, monolithic, integral connection, frictional engagement, mutual grooves, adhesive, raised elements, spikes, clips, snaps, friction fittings, compressive fittings, expanding rivets, staples, fixation plates, key/keyslot, tongue in groove, dovetail, magnetic connection and/or posts.

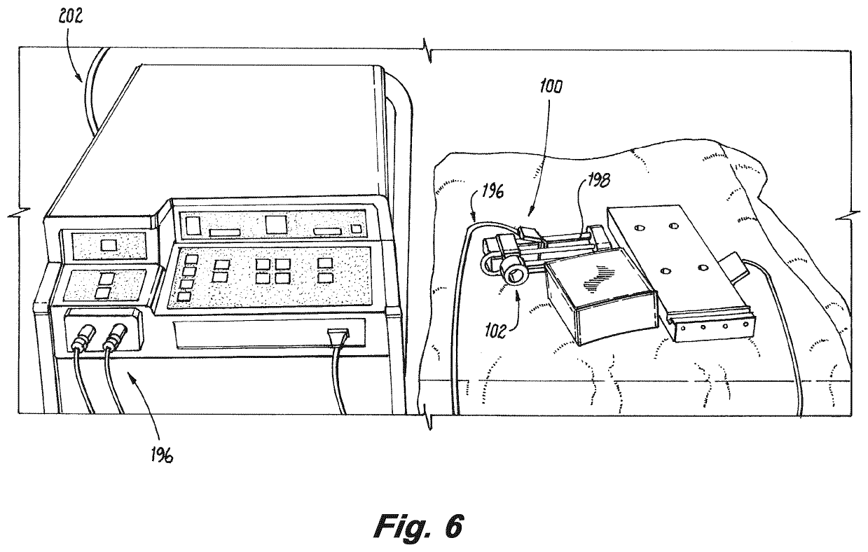

Resection device 102 includes a connector, such as, for example, a lead 196 having a first end 198 that is coupled to second conductive portion 134 of shaft 124 and an opposite second end 200 that is configured to be coupled to a power supply, such as, for example, a bipolar power supply 202 of surgical system 100, as shown in FIG. 6, such that electric power (positive and/or negative voltages) produced by power supply 202 is transmitted to second conductive portion 134 of shaft 124. The electrical power from lead 196 conducts through second roller 144 and/or hub 148 from second conductive portion 134 and from second roller 144 and/or hub 148 through tread 150. In some embodiments, tread 150 and second roller 144 are each made from at least in part from a conductive material such that electric power from tread 150 conducts through first roller 142; the electric power from lead 196 thus conducts through each of first roller 142, second roller 144 and tread 150 to cauterize and/or seal tissue, as discussed herein.

Resection device 102 further includes a connector, such as, for example, a lead 197 having a first end 199 that is coupled to first conductive portion 176 of shaft 156 and an opposite second end 201 that is configured to be coupled to a power supply, such as, for example, bipolar power supply 202 such that electric power (positive and/or negative voltages) produced by bipolar power supply 202 is transmitted to first conductive portion 176 of shaft 156. The electrical power from lead 197 conducts through second roller 188 and/or hub 192 and from second roller 188 and/or hub 192 through tread 194. In some embodiments, tread 194 and second roller 188 are each made from at least in part from a conductive material such that electric power from tread 194 conducts through first roller 186; the electric power from lead 197 thus conducts through each of first roller 186, second roller 188 and tread 194 to cauterize and/or seal tissue, as discussed herein.

Power supply 202 is configured to supply electrical energy such that the electrical energy conducts through first and second roller assemblies 122, 154 to cauterize and/or seal tissue positioned between treads 150, 194. Power supply 202 can be a bipolar power supply that may be adapted to output both positive and negative polarities, or both source and sink current. As such, it is envisioned that second end 200 of lead 196 may be connected to an output of power supply 202 that outputs positive polarities and thus acts as a source current and that second end 201 of lead 197 may be connected to an output of power supply 202 that outputs negative polarities and thus acts as a sink current. When tissue is positioned between treads 150, 194, the tissue becomes a conductor that conducts the electrical energy from tread 150 through the tissue to tread 194. The electrical energy then conducts through roller 186 and/or roller 188 to lead 197. As such, lead 197 acts as a ground such that the positive voltages of electrical energy from lead 196 conduct through first and second roller assemblies 122, 154 to lead 197; and from lead 197 to power supply 202. Alternatively, it is envisioned that second end 201 of lead 197 may be connected to an output of power supply 202 that outputs positive polarities and thus acts as a source current and that second end 200 of lead 196 may be connected to an output of power supply 202 that outputs negative polarities and thus acts as a sink current. When tissue is positioned between treads 150, 194, the tissue becomes a conductor that conducts the electrical energy from tread 150 through the tissue to tread 194. The electrical energy then conducts through roller 142 and/or roller 144 to lead 196. As such, lead 196 acts as a ground such that the positive voltages of electrical energy from lead 197 conduct through first and second roller assemblies 122, 154 to lead 196; and from lead 196 to power supply 202. However, whether lead 196 or lead 197 is connected to the positive output of power supply 202 (and the other of lead 196 and lead 197 is connected to the negative output of power supply 202, the outputs of power supply 202 that leads 196, 197 are connected to function in forced continuous conduction mode to allow resection device 102 to continuously provide electrical energy to roller assemblies 122, 154 to cauterize and/or seal tissue between treads 150, 194.

In assembly, operation and use, surgical system 100 is employed with a surgical procedure, such as, for example, a treatment of an applicable condition or injury of an affected section of an intestine. In some embodiments, one or all of the components of surgical system 100 can be delivered or utilized as a pre-assembled device. Surgical system 100 may be completely or partially revised, removed or replaced.

In use, to treat an intestine via a bowel resection, for example, a medical practitioner obtains access to a surgical site in any appropriate manner, such as through incision and retraction of tissues. In some embodiments, surgical system 100 can be used in any existing surgical method or technique including open surgery, mini-open surgery, minimally invasive surgery and percutaneous surgical implantation, whereby the intestine is accessed through a mini-incision, or sleeve that provides a protected passageway to the area. Once access to the surgical site is obtained, the particular surgical procedure can be performed for treating the intestine.

An incision is made in the body of a patient and a cutting instrument creates a surgical pathway for surgical system 100. A preparation instrument can be employed to prepare tissue surfaces of the tissue, intestine as an example, as well as for aspiration and irrigation of a surgical region. In some embodiments, target tissue T of the patient's intestine is identified by the medical practitioner. Resection device 102 is manipulated for positioning of resection device 102 such that target tissue T is positioned between tread 150 and tread 194. In some embodiments, the distance between tread 150 and tread 194 is increased to position target tissue T between tread 150 and tread 194 by rotating screw 168 in the direction shown by arrow A in FIG. 4 such that second shaft 156 translates relative to first shaft 124 and/or housing 104 in the direction shown by arrow B in FIG. 2. Increasing the distance between tread 150 and tread 194 allows ample space between tread 150 and tread 194 for target tissue T to be inserted between tread 150 and tread 194.

Once target tissue T is inserted between tread 150 and tread 194, target tissue T may be compressed between tread 150 and tread 194 by rotating screw 168 relative to housing 104 in the direction shown by arrow C in FIG. 5 such that second shaft 156 translates relative to first shaft 124 and/or housing 104 in the direction shown by arrow D in FIG. 2. In some embodiments, screw 168 is rotated relative to housing 104 such that second shaft 156 translates relative to first shaft 124 and/or housing 104 in a manner that compresses target tissue T with a force of about 190 N. In some embodiments, electrical current from bipolar power supply 202 conducts continuously through leads 196, 197 as screw 168 is rotated relative to housing 104 to compress target tissue T between treads 150, 194 such that the electrical current conducts continuously through rollers 142, 144, 186, 188 and treads 150, 194, as discussed herein, as target tissue T is being compressed between treads 150, 194. In some embodiments, electrical current from bipolar power supply 202 is applied continuously through leads 196, 197 after screw 168 is rotated relative to housing 104 to compress target tissue T between treads 150, 194 such that the electrical current conducts continuously through rollers 142, 144, 186, 188 and treads 150, 194, as discussed herein, after target tissue T is compressed between treads 150, 194.

In some embodiments, treads 150, 194 may be translated across target tissue T as the electrical current conducts continuously through rollers 142, 144, 186, 188 and treads 150, 194 to continuously cauterize and/or seal target tissue T. In some embodiments, first and second rollers 142, 144 can rotate relative to shaft 124 and first and second rollers 186, 188 can rotate relative to shaft 156 via free rotation of first and second rollers 142, 144 relative to shaft 124 and/or via manipulation of first and second rollers 142, 144 by hand as treads 150, 194 are translated across target tissue T. That is, first and second rollers 142, 144 can rotate relative to shaft 124 and first and second rollers 186, 188 can rotate relative to shaft 156 by medical practitioner manipulating resection device 102 relative to the patient's body such that treads 150, 194 translate across target tissue T. In some embodiments wherein resection device includes hubs 146, 148, 190, 192, the motors of hubs 146, 148, 190, 192 may be actuated to rotate first and second rollers 142, 144 relative to shaft 124 and first and second rollers 186, 188 relative to shaft 156 as treads 150, 194 are translated across target tissue T, as discussed herein.

In some embodiments, system 100 is configured to provide feedback to the medical practitioner to determine the amount or level of cauterization and/or sealing of tissue, such as, for example, target tissue T. As would be apparent to one of ordinary skill in the art, such feedback can be beneficial to allow the medical practitioner to know if tissue, such as, for example, target tissue T is sufficiently cauterized and/or sealed. Accordingly, in some embodiments, system includes one or more devices that are configured to function with resection device 102 to measure various parameters, such as, for example, impedance, temperature, etc. to provide feedback to the medical practitioner to determine the amount or level of cauterization and/or sealing. In particular, system 100 can include an electrical device, such as, for example, a smart generator that is adapted to be connected to resection device 102. The smart generator is a power supply, which has the capability to measure various input parameters such as temperature, voltage, power, tissue impedance, force, distance (gap) or speed of movement and control energy output. It is envisioned that the smart generator may be connected to resection device 102 wirelessly or via a wired connection. The smart generator may singularly produce RF energy, ultrasonic energy, or an electrical output to support a resistive heating element in the smart generator, or a combination of two or more of these modalities. The smart generator can include a means of providing feedback to the physician, such as, for example, a display that provides information such as a progress bar, temperature, and/or Watts, an audible tone which varies, or tactile output transmitted through resection device 102.

During operation of resection device 102, power supply 202 provides continuous electrical energy such that the electrical energy conducts continuously through first and second roller assemblies 122, 154 to continuously cauterize and/or seal target tissue T between treads 150, 194. The physician may translate treads 150, 194 across target tissue T as resection device 102 continuously cauterizes and/or seals target tissue T, as discussed herein. Prior to continuously cauterizing and/or sealing target tissue T using resection device 102, the smart generator may measure various parameters of target tissue T such as, but not limited to, the temperature, tissue impedance and mechanical compliance of target tissue T. These parameters are used by the smart generator to determine the initial output parameters of power supply 202 and begin energy delivery by continuously conducting energy through leads 196, 197 and first and second roller assemblies 122, 154 to continuously cauterize and/or seal target tissue T between treads 150, 194. This energy delivery may be increased in a gradual manner, until the energy conditions measured as optimal to initiate the tissue effect, and feedback may be provided to the physician that conditions have been met to support proper treatment, and the advancement of resection device 102 through target tissue T.

The physician then begins advancing resection device 102 through target tissue T by translating treads 150, 194 along target tissue T, as discussed above. After the initial activation, the smart generator begins a continuous cycle of sensing, processing, and adjusting the energy output of power supply 202 to maintain or achieve a desired state for the creation of hemostasis as resection device 102 is advanced by the physician. For instance, in some embodiments, a closed-loop feedback system in the smart generator can measure the temperature and impedance input parameters, interpret this data, and modify the output of power supply 202. The rate of change of these input parameters may be monitored, and this may be used to adjust the sensitivity of the feedback system, for instance the initial proportional, integral, differential (PID) setting may be adjusted to allow the closed-loop feedback system to adapt more effectively. The feedback system may also provide output to the user to support the functionality of resection device 102, by giving information to the user that must stay within a range. For instance, the measured temperature of resection device 102 may be displayed, and the user adjusts the rate of movement of resection device 102 through target tissue T to not go below a specified level. This process allows the closed-loop feedback system to self-regulate and adapt to changes in target tissue T, ensuring stability and optimal performance.

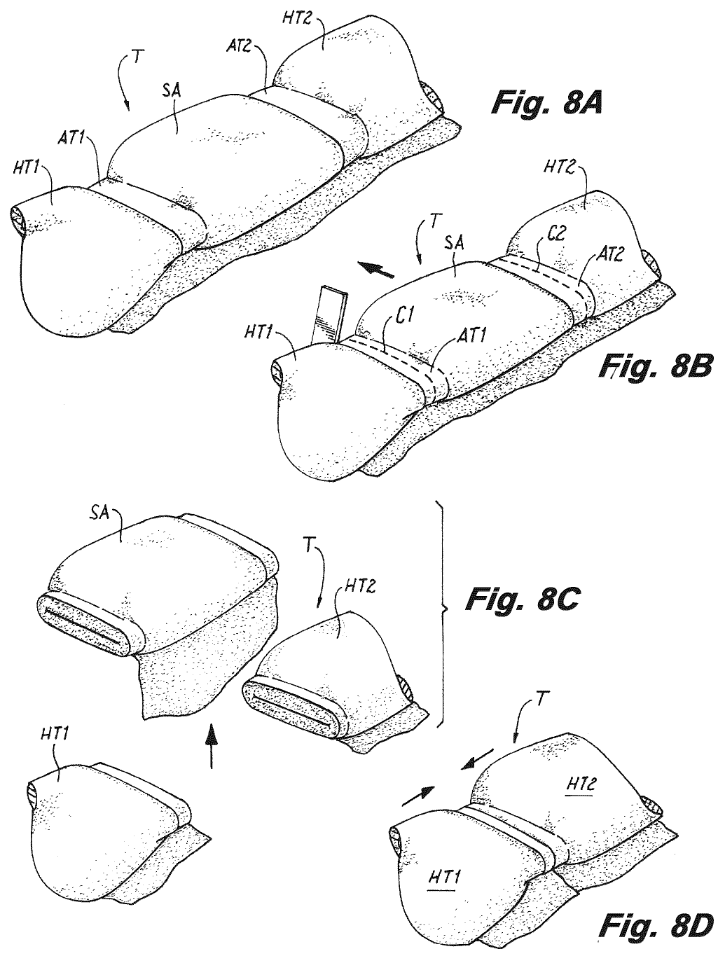

Continuous energy delivery from resection device 102 to target tissue T cauterizes blood vessels, lymphatic vessels, and cells. Upon achieving successful treatment of target tissue T, appropriate clinical steps will follow to ensure hemostasis. The surgical team will then evaluate next steps in patient care and proceed accordingly. For example, in some clinical situations, this can include intestinal resection, which involves, among other things, connecting the cut ends of bowel. Subsequently, routine clinical care would involve testing the connection, then proceeding through routine steps in closing the abdominal incision. In particular, in treating target tissue T, it may be deemed beneficial to separate a selected area SA of target tissue T a cutting mechanism to remove pathologic tissue from healthy tissue. For example, in some embodiments, resection device 102 may be used to create spaced apart first and second areas of altered tissue AT1 and AT2 that are created by translating treads 150, 194 across target tissue T, as shown in FIG. 8A. In some embodiments, first area of altered tissue AT1 is created by translating treads 150, 194 across target tissue T in a first step of the disclosed method and second area of altered tissue AT2 is created by translating treads 150, 194 across target tissue T in a second step of the disclosed method that is distinct from the first step such that areas of altered tissue AT1, AT2 are spaced apart by selected area SA of target tissue T. In some embodiments, selected area SA of target tissue T is spaced apart from a first area of healthy tissue HT1 by first area of altered tissue AT1 and selected area SA of target tissue T is spaced apart from a second area of healthy tissue HT2 by second area of altered tissue AT2, as shown in FIG. 8A. In some embodiments, selected area SA of target tissue includes a portion of target tissue that requires removal, wherein selected area SA is removed or separated first and second areas of healthy tissue HT1, HT2 so that first and second areas of healthy tissue HT1, HT2 may be joined, as discussed herein. In some embodiments, separation is accomplished by a fixed or movable cutting mechanism in some embodiments and laser energy in other embodiments. For example, in one embodiment, a blade may be inserted between treads 150, 194 as target tissue is positioned between treads 150, 194 to cut target tissue T between treads 150, 194 using the blade.

In some embodiments, resection device 102 includes a cutting mechanism, such as, for example, a blade assembly that is configured to cut cauterized tissue, such as, for example, target tissue T to separate first and second areas of healthy tissue HT1, HT2 from the selected area SA of healthy tissue T. In particular, the blade assembly can include a rotatable/retractable blade that has a cutting surface configured to cut cauterized tissue, for example. For example, it is envisioned that the blade can be rotatable relative to shaft 124 and/or second shaft 156 between a retracted orientation in which the blade extends parallel to longitudinal axis X, third non-conductive portion 140 of shaft 124 and/or second shaft 156, and an extended orientation, in which the blade extends perpendicular to longitudinal axis X, third non-conductive portion 140 of shaft 124 and/or second shaft 156. In some embodiments, the blade is moved from the retracted orientation to the extended orientation when cauterized tissue is positioned between treads 150, 194 to cut the cauterized tissue, as discussed herein.

In embodiments wherein resection device 102 includes the blade assembly, the blade assembly may be used to selectively cut target tissue T. For example, the blade may be moved from the retracted orientation to the extended orientation when first area of altered tissue AT1 is positioned between treads 150, 194 to create a first cut C1 in first area of altered tissue AT1, as shown in FIG. 8B. Likewise, the blade may be moved from the retracted orientation to the extended orientation when second area of altered tissue AT2 is positioned between treads 150, 194 to create a second cut C2 in second area of altered tissue AT2, as shown in FIG. 8B. Following the creation of first and second cuts C1, C2 in first area of altered tissue AT1 and second area of altered tissue AT2, selected area SA of target tissue T can be separated from first and second areas of healthy tissue HT1, HT2, as shown in FIG. 8C. First and second areas of healthy tissue HT1, HT2 may then be joined to one another, as shown in FIG. 8D.

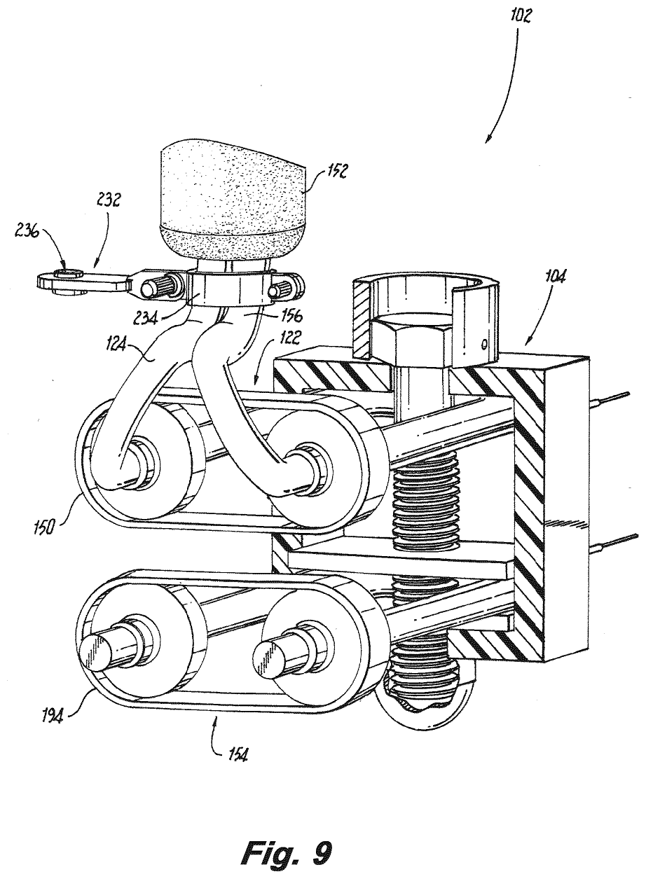

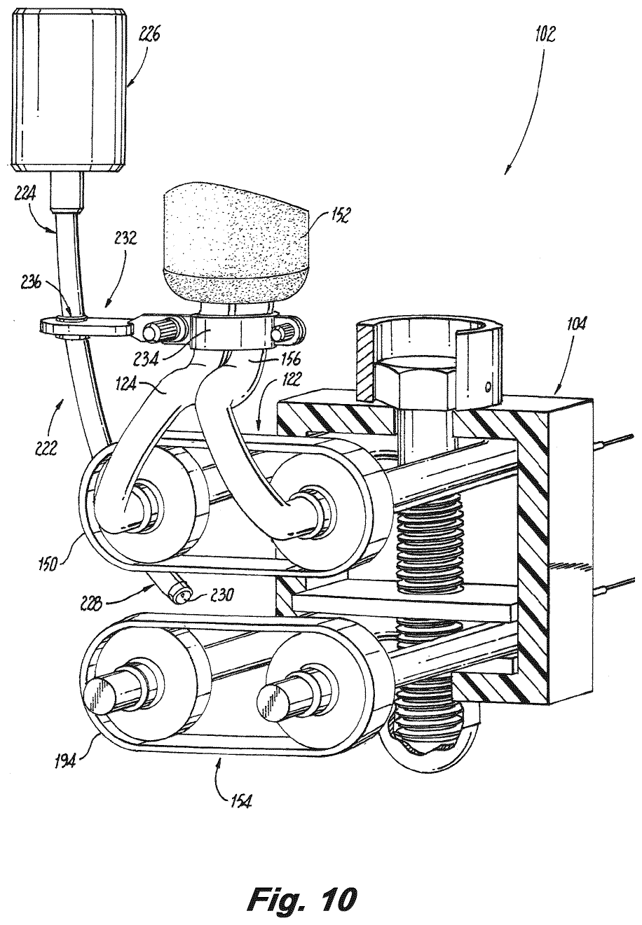

In some embodiments, surgical system 100 can include a device or component that is adapted to further assist in cauterizing tissue between treads 150, 194. For example, in one embodiment, shown in FIGS. 9 and 10, surgical system 100 includes a connector, such as, for example, a cable 222 having a first end 224 that is configured to be connected to a power supply 226 and an opposite second end 226 that is configured to be positioned adjacent to treads 150, 194. Second end 226 includes or is connected to a laser head 230. Laser head 230 includes one or a plurality of laser sources that are configured to produce laser energy, such as, for example, one or more laser beams, as shown in FIG. 10. In particular, the laser energy is emitted from second end 224 of cable 222 to direct the laser energy between treads 150, 194 to further assist in cauterizing tissue between treads 150, 194. That is, surgical system 100 can be adapted to provide laser energy to tissue between treads 150, 194 as electrical energy from rollers 142, 144, 186, 188 conducts through treads 150, 194 to cauterize the tissue with electrical and laser energy simultaneously. In some embodiments, to facilitate cauterizing the tissue with electrical and laser energy simultaneously, cable 222 may be adapted to be removably connected with resection device 102. For example, resection device 102 can include a clip, such as, for example, a bracket 232 that is coupled to a portion of resection device 102 such that second end 228 of cable 222 can be positioned to direct laser energy to the tissue positioned between treads 150, 194. It is envisioned that bracket 232 can be variously positioned relative to resection device 102, however, in one embodiment, bracket 232 is coupled to shaft 124 and/or shaft 156 adjacent to handle 152. In particular, bracket 232 includes a body 234 that is coupled to shaft 124 and/or shaft 156. Body 234 defines an opening 236 configured for disposal of cable 222 such that second end 228 of cable 222 can be inserted through opening 236 after first end 224 of cable 222 is coupled to power supply 226. In some embodiments, cable 222 is configured to be bent to position laser head 230 in a selected orientation and/or position relative to the tissue positioned between treads 150, 194. In some embodiments, cable 222 can be made from a deformable material that maintains the orientation and/or position of cable 222. In some embodiments, cable 222 is a fiber optic cable. In some embodiments, the laser source(s) of laser head 230 include(s) a polarization beam element and/or one or more light-emitting devices. In some embodiments, first end 224 of cable 222 may be connected to power supply 202 to provide energy to laser head 230. In some embodiments, bracket 232 can be variously connected with resection device 102, such as, for example, monolithic, integral connection, frictional engagement, threaded engagement, mutual grooves, screws, adhesive, nails, barbs, raised elements, spikes, clips, snaps, friction fittings, compressive fittings, expanding rivets, staples, fixation plates, key/keyslot, tongue in groove, dovetail, magnetic connection and/or posts.

In some embodiments, it is envisioned that the laser energy that is emitted from second end 224 of cable 222 can be used to cut target tissue T, as discussed herein. That is, system 100 may include a laser to cut target tissue T in place of or in addition to a blade assembly. For example, energy from laser head 230 may be directed to first area of altered tissue AT1 when first area of altered tissue AT1 is positioned between treads 150, 194 to create first cut C1 in first area of altered tissue AT1 and energy from laser head 230 may be directed to second area of altered tissue AT2 when second area of altered tissue AT2 is positioned between treads 150, 194 to create second cut C2 in second area of altered tissue AT2. As discussed above with regard to the blade assembly, following the creation of first and second cuts C1, C2 in first area of altered tissue AT1 and second area of altered tissue AT2, selected area SA of target tissue T can be separated from first and second areas of healthy tissue HT1, HT2, as shown in FIG. 8C. First and second areas of healthy tissue HT1, HT2 may then be joined to one another, as shown in FIG. 8D.

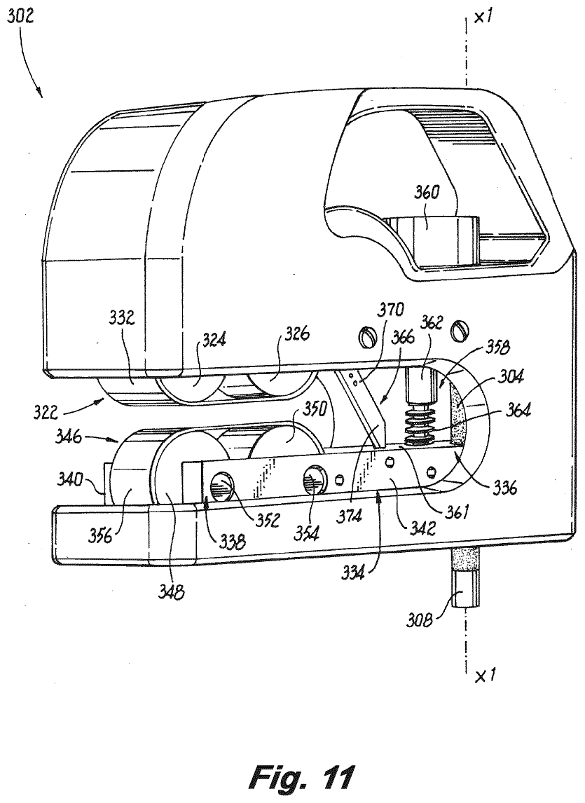

In some embodiments, shown in FIGS. 11-14, surgical system 100 includes a resection device 302 that is similar to resection device 102. Resection device 302 is an in-line, continuous resection instrument. Similar to resection device 102, resection device 302 is configured to cauterize tissue in an in-line fashion by compressing the tissue, translating linearly across the tissue and applying electrical energy in a continuous fashion to cauterize and/or seal the tissue, as discussed herein.

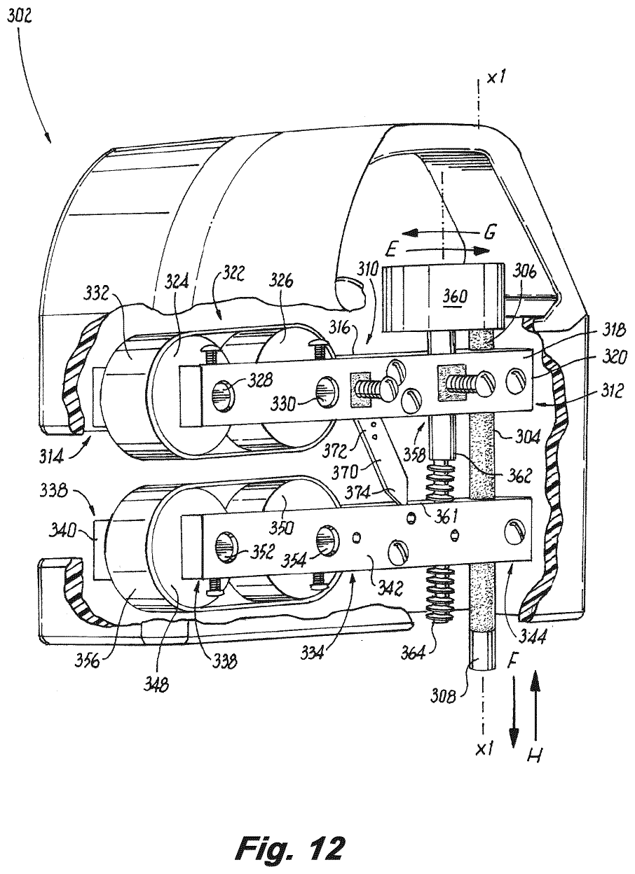

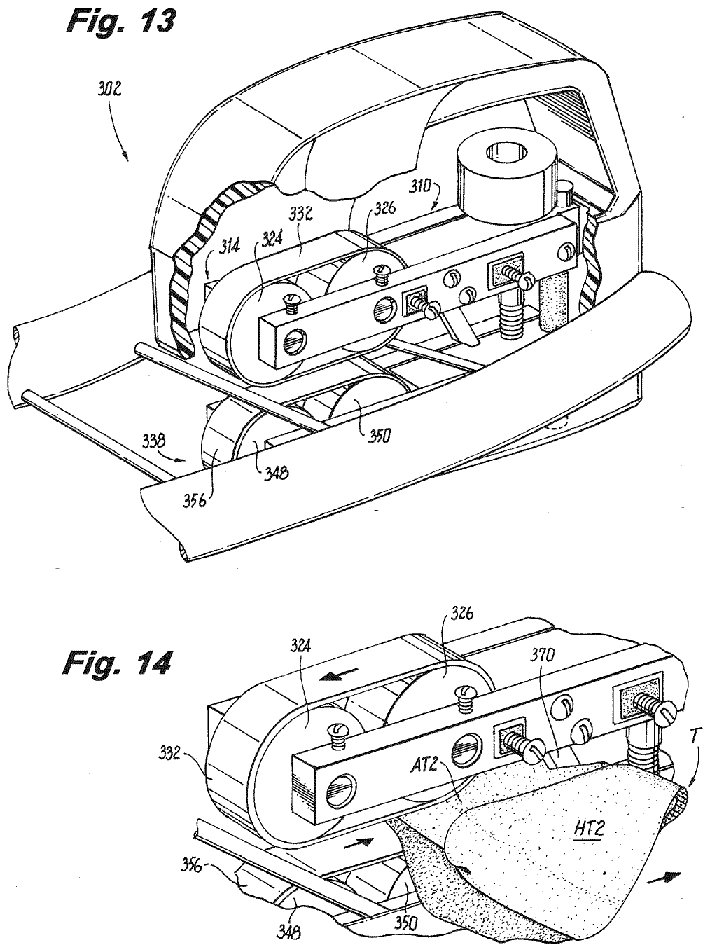

Resection device 302 includes a shaft 304 extending along a longitudinal axis X1 between a proximal end 306 and an opposite distal end 308, as best shown in FIGS. 12 and 13. Resection device 302 includes an extension, such as, for example, a first arm 310 having a first end 312 coupled to proximal end 306 of shaft 304 and an opposite second end 314. First arm 310 includes a first rail 316 and a second rail 318 that is spaced apart from first rail 316. Rails 316, 318 are coupled to shaft 304 by a pin 320 that extends through each of first rail 316, shaft 304 and second rail 318 such that rails 316, 318 each extend parallel to one another. In some embodiments, rails 316, 318 each extend perpendicular to shaft 304 and/or axis X1. In some embodiments, first rail 316 and/or second rail 318 may be disposed at alternate orientations, relative to shaft 304 and/or axis X1, such as, for example, transverse and/or other angular orientations such as acute or obtuse, co-axial and/or may be offset or staggered. In some embodiments, rails 316, 318 are each permanently fixed at an angle relative to shaft 304 and/or axis X1, such as, for example, a 90 degree angle relative to shaft 304 and/or axis X1. In some embodiments, first rail 316 and second rail 318 are rotatable relative to shaft 304 about pin 320 to alter the angle of rails 316, 318 relative to shaft 304 and/or axis X1. In some embodiments, first rail 316 and second rail 318 are rotatable relative to shaft 304 about pin 320 to alter the angle of rails 316, 318 relative to shaft 304 and/or axis X1 between an angle of about −45 degrees to about +45 degrees relative to shaft 304 and/or axis X1.

A first roller assembly 322 is coupled to second end 314 of first arm 310. First roller assembly 322 comprises a wheel, such as, for example, a first roller 324 and a wheel, such as, for example, a second roller 326 positioned between rails 316, 318 such that second roller 326 is in line with first roller 324. A pin 328 extends through first rail 316, first roller 324 and second rail 318 such that first roller 324 is rotatable relative to rails 316, 318. That is, first roller 324 is configured to rotate 360 degrees about pin 328 in opposite directions. Likewise, a pin 330 extends through first rail 316, second roller 326 and second rail 318 such that second roller 326 is rotatable relative to rails 316, 318. That is, second roller 326 is configured to rotate 360 degrees about pin 330 in opposite directions. In some embodiments, first roller 324 and/or second roller 326 are configured to rotate freely relative to rails 316, 318 about pin 328 and pin 330, respectively. That is, first roller 324 and/or second roller 326 are configured to be rotated relative to rails 316, 318 manually. In some embodiments, first roller 324 and/or second roller 326 are configured to rotate relative to rails 316, 318 via automation. For example, in some embodiments, roller assembly 322 includes a hub similar to hub 146 that is disposed about pin 328 and first roller 324 is disposed about the hub. The hub can include an actuator, such as, for example, a motor that is configured to rotate first roller 324 about the hub for rotation of first roller 324 about pin 328. Likewise, in some embodiments, roller assembly 322 includes a hub that is similar to hub 148 that is disposed about pin 330 and second roller 326 is disposed about the hub for rotation of second roller 326 about pin 330. The hub can include an actuator, such as, for example, a motor that is configured to rotate second roller 326 about the hub for rotation of second roller 326 about pin 330.

First roller assembly 322 includes a band, such as, for example, a tread 332 extending about first roller 324 and second roller 326. Tread 332 is configured for rotation relative to rails 316, 318. That is, tread 332 is positioned about first roller 324 and second roller 326 such that rotation of first roller 324 and second roller 326 relative to rails 316, 318 also rotates tread 332 relative to rails 316, 318. Tread 332 is an electrode and is made at least in part from a material that is electrically conductive such that electrical current from the first roller 324 and/or the second roller 326 will conduct through tread 332, as discussed herein. In some embodiments, tread 332 includes electrically conductive tape. In some embodiments, tread 332 includes one or a plurality of layers. In some embodiments, tread 322 has a band or loop configuration and extends continuously about first roller 324 and second roller 326. In some embodiments, tread 332 extends about first roller 324 and second roller 326 in a manner that prevents relative movement between tread 332 and first roller 324 and/or second roller 326. In some embodiments, tread 332, first roller 324 and/or second roller 326 may have various surface configurations to enhance fixation of tread 332 with first roller 324 and/or second roller 326, such as, for example, rough, arcuate, undulating, porous, semi-porous, dimpled, polished and/or textured according to the requirements of a particular application. In some embodiments, tread 332 can be variously connected with first roller 324 and/or second roller 326, such as, for example, monolithic, integral connection, frictional engagement, mutual grooves, adhesive, raised elements, spikes, clips, snaps, friction fittings, compressive fittings, expanding rivets, staples, fixation plates, key/keyslot, tongue in groove, dovetail, magnetic connection and/or posts.

Resection device 302 includes an extension, such as, for example, a second arm 334 having a first end 336 coupled to distal end 308 of shaft 304 and an opposite second end 338. Second arm 334 includes a first rail 340 and a second rail 342 that is spaced apart from first rail 340. Rails 340, 342 are coupled to shaft 304 by a pin 344 that extends through each of first rail 340, shaft 304 and second rail 342 such that rails 340, 342 each extend parallel to one another. In some embodiments, rails 340, 342 each extend perpendicular to shaft 304 and/or axis X1. In some embodiments, rails 340, 342 each extend parallel to rails 316, 318. In some embodiments, first rail 340 and/or second rail 342 may be disposed at alternate orientations, relative to shaft 304 and/or axis X1, such as, for example, transverse and/or other angular orientations such as acute or obtuse, co-axial and/or may be offset or staggered. In some embodiments, rails 340, 342 are each permanently fixed at an angle relative to shaft 304 and/or axis X1, such as, for example, a 90 degree angle relative to shaft 304 and/or axis X1. In some embodiments, first rail 340 and second rail 342 are rotatable relative to shaft 304 about pin 344 to alter the angle of rails 340, 342 relative to shaft 304 and/or axis X1. In some embodiments, first rail 340 and second rail 342 are rotatable relative to shaft 304 about pin 344 to alter the angle of rails 340, 342 relative to shaft 304 and/or axis X1 between an angle of about −45 degrees to about +45 degrees relative to shaft 304 and/or axis X1. In some embodiments, rails 316, 318 rotate relative to shaft 304 and/or axis X1 about pin 320 in unison as rails 340, 342 rotate relative to shaft 304 and/or axis X1 about pin 344 such that arm 334 remains parallel to arm 310 during rotation of arms 310, 334 relative to shaft 304 and/or axis X1 about pins 320, 344.

A second roller assembly 346 is coupled to second end 338 of second arm 334. Second roller assembly 346 comprises a wheel, such as, for example, a first roller 348 and a wheel, such as, for example, a second roller 350 positioned between rails 340, 342 such that second roller 350 is in line with first roller 348. A pin 352 extends through first rail 340, first roller 348 and second rail 342 such that first roller 348 is rotatable relative to rails 340, 342. That is, first roller 348 is configured to rotate 360 degrees about pin 352 in opposite directions. Likewise, a pin 354 extends through first rail 340, second roller 350 and second rail 342 such that second roller 350 is rotatable relative to rails 340, 342. That is, second roller 350 is configured to rotate 360 degrees about pin 354 in opposite directions. In some embodiments, first roller 348 and/or second roller 350 are configured to rotate freely relative to rails 340, 342 about pin 352 and pin 354, respectively. That is, first roller 348 and/or second roller 350 are configured to be rotated relative to rails 340, 342 manually. In some embodiments, first roller 348 and/or second roller 350 are configured to rotate relative to rails 340, 342 via automation. For example, in some embodiments, roller assembly 346 includes a hub similar to hub 190 that is disposed about pin 352 and first roller 348 is disposed about the hub. The hub can include an actuator, such as, for example, a motor that is configured to rotate first roller 348 about the hub for rotation of first roller 348 about pin 352. Likewise, in some embodiments, roller assembly 346 includes a hub that is similar to hub 192 that is disposed about pin 354 and second roller 350 is disposed about the hub for rotation of second roller 350 about pin 354. The hub can include an actuator, such as, for example, a motor that is configured to rotate second roller 350 about the hub for rotation of second roller 350 about pin 354.

Second roller assembly 322 includes a band, such as, for example, a tread 356 extending about first roller 348 and second roller 350. Tread 356 is configured for rotation relative to rails 340, 342. That is, tread 356 is positioned about first roller 348 and second roller 350 such that rotation of first roller 348 and second roller 350 relative to rails 340, 342 also rotates tread 356 relative to rails 340, 342. Tread 356 is an electrode and is made at least in part from a material that is electrically conductive such that electrical current from the first roller 348 and/or the second roller 350 will conduct through tread 356, as discussed herein. In some embodiments, tread 356 includes electrically conductive tape. In some embodiments, tread 356 includes one or a plurality of layers. In some embodiments, tread 356 has a band or loop configuration and extends continuously about first roller 348 and second roller 350. In some embodiments, tread 356 extends about first roller 348 and second roller 350 in a manner that prevents relative movement between tread 356 and first roller 348 and/or second roller 350. In some embodiments, tread 356, first roller 348 and/or second roller 350 may have various surface configurations to enhance fixation of tread 356 with first roller 348 and/or second roller 350, such as, for example, rough, arcuate, undulating, porous, semi-porous, dimpled, polished and/or textured according to the requirements of a particular application. In some embodiments, tread 356 can be variously connected with first roller 348 and/or second roller 350, such as, for example, monolithic, integral connection, frictional engagement, mutual grooves, adhesive, raised elements, spikes, clips, snaps, friction fittings, compressive fittings, expanding rivets, staples, fixation plates, key/keyslot, tongue in groove, dovetail, magnetic connection and/or posts.

As shown in FIGS. 11-13, tread 356 can be spaced apart from tread 332 to define an opening between treads 332, 356 configured for disposal of a material, such as, for example, tissue. This allows the tissue to be positioned between treads 332, 356, as shown in FIG. 14, such that electrical energy conducting through rollers 324, 326, 348, 350 and/or treads 332, 356 to cauterize and/or seal the tissue, as discussed herein. In some embodiments, resection device 302 includes an actuator, such as, for example, a screw 358 that is configured to increase and/or decrease the distance between treads 332, 356 to increase the size of the opening defined by the space between treads 332, 356 to accommodate tissue or other anatomy of different thicknesses and/or to compress the tissue. In particular, resection device 302 includes a first part, such as, for example, a first plate 359 and a second part, such as, for example, a second plate 361. First plate 359 is positioned between rails 316, 318 such that first plate 359 is fixed relative to rails 316, 318 and second plate 361 is positioned between rails 340, 342 such that second plate 361 is fixed relative to rails 340, 342. Screw 358 includes a head 360, a non-threaded portion 362 connected to head 360 and a threaded portion 364 connected to non-threaded portion 362. Non-threaded portion 362 extends through first plate 359 and threaded portion 374 extends through second plate 361 such that rotation of screw 358 relative to arms 310, 334 and/or plates 359, 361 in a first rotational direction, such as, for example, the direction shown by arrow E in FIG. 12, translates arm 334 relative to arm 310 and/or shaft 304 in the direction shown by arrow F in FIG. 12 along axis X1; and rotation of screw 358 relative to arms 310, 334 and/or plates 359, 361 in an opposite second rotational direction, such as, for example, the direction shown by arrow G in FIG. 12, translates arm 334 relative to arm 310 and/or shaft 304 in the direction shown by arrow H in FIG. 12 along longitudinal axis X1. In some embodiments, first plate 359 can be variously connected with rails 316, 318 and/or second plate 361 can be variously connected with rails 340, 342, such as, for example, monolithic, integral connection, frictional engagement, threaded engagement, mutual grooves, screws, adhesive, nails, barbs, raised elements, spikes, clips, snaps, friction fittings, compressive fittings, expanding rivets, staples, fixation plates, key/keyslot, tongue in groove, dovetail, magnetic connection and/or posts.

Resection device 302 includes a connector, such as, for example, lead 196 wherein first end 198 is coupled to pin 328 and/or pin 330 and an opposite second end 200 that is configured to be coupled to power supply 202 such that electric power (positive and/or negative voltages) produced by power supply 202 is transmitted to pin 328 and/or pin 330. The electrical power from pin 328 and/or pin 330 conducts through first roller 322 and/or second roller 324 and from first roller 322 and/or second roller 324 through tread 332. In some embodiments, pin 328, pin 330, roller 324, roller 326 and tread 332 are each made from at least in part from a conductive material.

Resection device 302 further includes a connector, such as, for example, lead 197 wherein first end 199 is coupled to pin 352 and/or pin 354 and second end 201 is configured to be coupled to power supply 202 such that electric power (positive and/or negative voltages) produced by bipolar power supply 202 is transmitted to pin 352 and/or pin 354. The electrical power from pin 352 and/or pin 354 conducts through first roller 348 and/or second roller 350 and from first roller 348 and/or second roller 350 through tread 356. In some embodiments, pin 352, pin 354, roller 3248, roller 3250 and tread 356 are each made from at least in part from a conductive material.

Power supply 202 is configured to supply electrical energy such that the electrical energy conducts through first and second roller assemblies 322, 346 to cauterize and/or seal tissue positioned between treads 332, 356. As discussed above, power supply 202 can be a bipolar power supply that may be adapted to output both positive and negative polarities, or both source and sink current. As such, it is envisioned that second end 200 of lead 196 may be connected to an output of power supply 202 that outputs positive polarities and thus acts as a source current and that second end 201 of lead 197 may be connected to an output of power supply 202 that outputs negative polarities and thus acts as a sink current. When tissue is positioned between treads 332, 356, the tissue becomes a conductor that conducts the electrical energy from tread 332 through the tissue to tread 356. The electrical energy then conducts through roller 348 and/or roller 350 to lead 197. As such, lead 197 acts as a ground such that the positive voltages of electrical energy from lead 196 conduct through first and second roller assemblies 322, 346 to lead 197; and from lead 197 to power supply 202. Alternatively, it is envisioned that second end 201 of lead 197 may be connected to an output of power supply 202 that outputs positive polarities and thus acts as a source current and that second end 200 of lead 196 may be connected to an output of power supply 202 that outputs negative polarities and thus acts as a sink current. When tissue is positioned between treads 332, 356, the tissue becomes a conductor that conducts the electrical energy from tread 356 through the tissue to tread 332. The electrical energy then conducts through roller 322 and/or roller 324 to lead 196. As such, lead 196 acts as a ground such that the positive voltages of electrical energy from lead 197 conduct through first and second roller assemblies 322, 356 to lead 196; and from lead 196 to power supply 202. However, whether lead 196 or lead 197 is connected to the positive output of power supply 202 (and the other of lead 196 and lead 197 is connected to the negative output of power supply 202, the outputs of power supply 202 that leads 196, 197 are connected to function in forced continuous conduction mode to allow resection device 302 to continuously provide electrical energy to roller assemblies 322, 346 to cauterize and/or seal tissue between treads 322, 356.

In some embodiments, resection device 302 includes a cutting mechanism, such as, for example, a blade assembly 366 that is similar to the blade assembly discussed above and is configured to cut cauterized tissue, for example. Blade assembly 366 includes a bracket 368 that is positioned between rails 316, 318 such that bracket 368 is fixed relative to rails 316, 318. Blade assembly 366 includes a blade 370 having a first end 372 connected with bracket 368 and an opposite second end 374. Second end 374 includes a cutting surface configured to cut cauterized tissue, for example. Blade assembly 366 includes a pin 376 extending through first end 372 of blade 370 and bracket 368 such that blade 370 is rotatable relative to bracket 368. In particular, blade 370 is rotatable relative to bracket 368 about pin 376 between a retracted orientation, in which blade 370 extends parallel to longitudinal axis X1, and an extended orientation, in which blade 370 extends perpendicular to longitudinal axis X1. In some embodiments, blade 370 is moved from the retracted orientation to the extended orientation when cauterized tissue is positioned between treads 332, 356 to cut the cauterized tissue, as discussed herein. In some embodiments, bracket 368 can be variously connected with rails 340, 342, such as, for example, monolithic, integral connection, frictional engagement, threaded engagement, mutual grooves, screws, adhesive, nails, barbs, raised elements, spikes, clips, snaps, friction fittings, compressive fittings, expanding rivets, staples, fixation plates, key/keyslot, tongue in groove, dovetail, magnetic connection and/or posts. In some embodiments, bracket 368 is movable relative to rails 316, 318. For example, in some embodiments, bracket 368 is configured to translate along rails 316, 318 toward and away from shaft 304.

Similar to resection device 102, resection device 302 may be used to continuously cauterize or seal tissue, such as, for example, target tissue T in a resection procedure, for example. In particular, in some embodiments, resection device 302 may be used to create spaced apart first and second areas of altered tissue AT1 and AT2 that are created by translating treads 332, 356 across target tissue T. In some embodiments, first area of altered tissue AT1 is created by translating treads 332, 356 across target tissue T in a first step of the disclosed method and second area of altered tissue AT2 is created by translating treads 332, 356 across target tissue T in a second step of the disclosed method that is distinct from the first step such that areas of altered tissue AT1, AT2 are spaced apart by selected area SA of target tissue T. In some embodiments, separation of selected area SA of target tissue T from first and second areas of healthy tissue HT1, HT1 is accomplished by a fixed or movable cutting mechanism in some embodiments and laser energy in other embodiments. For example, in one embodiment, blade 370 may be inserted between treads 332, 356 as target tissue T is positioned between treads 332, 356 to cut target tissue T between treads 332, 356 using blade 370.

In embodiments wherein resection device 302 includes blade assembly 366, blade assembly 366 may be used to selectively cut target tissue T. For example, blade 370 may be moved from the retracted orientation discussed herein to the extended orientation discussed when first area of altered tissue AT1 is positioned between treads 332, 356 to create first cut C1 in first area of altered tissue AT1 and blade 370 may be moved from the retracted orientation discussed herein to the extended orientation discussed herein when second area of altered tissue AT2 is positioned between treads 332, 356 to create second cut C2 in second area of altered tissue AT2. Following the creation of first and second cuts C1, C2 in first area of altered tissue AT1 and second area of altered tissue AT2, selected area SA of target tissue T can be separated from first and second areas of healthy tissue HT1, HT2. First and second areas of healthy tissue HT1, HT2 may then be joined to one another. It is envisioned that resection device 302 may be used in conjunction with an electrical device, such as, a smart generator, as discussed herein to provide feedback to the medical practitioner to determine the amount or level of cauterization and/or sealing, as also discussed herein.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical method, comprising:
providing resection device comprising:
a first part, and
a second part that is spaced apart from the first part to define an opening therebetween;
positioning a material within the opening;
translating the second part relative to the first part to compress the material;
continuously applying electrical energy to the first part and the second part; and
rotating the second part relative to the first part while applying electrical energy to the first part and the second part to cauterize the material.

2. The method recited in claim 1, wherein the material includes biological tissue.

3. The method recited in claim 1, wherein the material includes an organ of a living organism.

4. The method recited in claim 1, wherein the material includes a first piece of material and a second piece of material.

5. The method recited in claim 4, wherein cauterizing the material seals the first piece of material with the second piece of material.

6. The method recited in claim 1, wherein the material includes a first section of a bowel and a second section of the bowel.

7. The method recited in claim 6, wherein cauterizing the material seals the first section of the bowel with the second section of the bowel.

8. The method recited in claim 1, wherein the first part is a first pair of rollers and the second part is a second pair of rollers.

9. The method recited in claim 8, wherein the resection device further comprises a first tread extending about the first pair of rollers and a second tread extending about the second pair of rollers.

10. The method recited in claim 1, wherein the second part is translated relative to the first part to compress the material with a force of about 100 N to about 300 N.

11. The method recited in claim 1, wherein the electrical energy that is continuously applied is produced by an electrical conditioning and supply unit.

12. The method recited in claim 1, further comprising directing laser energy to the material.

13. The method recited in claim 1, further comprising:
coupling a source of laser energy to the resection device; and
directing laser energy to the material.

14. The method in claim 1, further comprising staples delivered into the material

15. A resection device, comprising:
a first part;
a second part that is rotatable relative to the first part, the second part being spaced apart from the first part to define an opening therebetween,
wherein the second part is configured to translate relative to the first part to compress a material positioned within the opening, and
wherein the first part and the second part are configured to continuously receive electrical energy to cauterize the material as the second part rotates relative to the first part.

16. The resection device recited in claim 15, wherein the first part is a first pair of rollers and the second part is a second pair of rollers.

17. The resection device recited in claim 16, further comprising a first tread extending about the first pair of rollers and a second tread extending about the second pair of rollers.

18. The resection device recited in claim 17, wherein the treads include conductive metal tape.

19. The resection device recited in claim 15, wherein the second part is configured to translate relative to the first part to compress the material with a force of about 100 N to about 300 N.

20. A surgical method, comprising:
providing a resection device, the resection device comprising:
a first part including a first pair of rollers and a first tread extending about the first pair of rollers,
a second part including a second pair of rollers and a second tread extending about the second pair of rollers, the second part being spaced apart from the first part to define an opening therebetween;
positioning a material within the opening;
translating the second part relative to the first part to compress the material;
continuously applying electrical energy to the first part and the second part; and
rotating the second part relative to the first part while applying electrical energy to the first part and the second part to cauterize the material,
wherein the material includes a first section of a bowel and a second section of the bowel,
wherein cauterizing the material seals the first section of the bowel with the second section of the bowel,
wherein the second part is translated relative to the first part to compress the material with a force of about 100 N to about 300 N, and
wherein the electrical energy that is continuously applied is produced by an electrical conditioning and supply unit.
